(19) United States
(12) Patent Application Publication
(10) Pub. No.: US 2023/0346220 A1
(43) Pub. Date: Nov. 2, 2023

(54) CONTACTLESS VITAL SIGNS MONITORING SYSTEM

(71) Applicant: Hangzhou Normal University Qianjiang College, Hangzhou (CN)

(72) Inventors: Shihua Cao, Hangzhou (CN); Yuchao Le, Hangzhou (CN); Zheng Chen, Hangzhou (CN); Danni He, Hangzhou (CN); Yanfei Chen, Hangzhou (CN); Beiying Qian, Hangzhou (CN)

(21) Appl. No.: 18/306,330

(22) Filed: Apr. 25, 2023

(30) Foreign Application Priority Data
Apr. 28, 2022 (CN) 2022104697141

(51) Int. Cl. A61B 5/00; A61B 5/11; G05B 19/042; H05B 47/16; H05B 47/11; H05B 47/115

(52) U.S. Cl. CPC A61B 5/0022; A61B 5/6892; A61B 5/1102; A61B 5/7225; A61B 5/721; A61B 5/4818; G05B 19/042; H05B 47/16; H05B 47/11; H05B 47/115; A61B 2562/0247; A61B 2560/0252; A61B 2562/029; G05B 2219/23153

(57) ABSTRACT

A contactless vital signs monitoring system is installed on a mattress and includes a contactless vital signs signal acquisition and processing system. The contactless vital signs signal acquisition and processing system includes a BCG signal acquisition module, a human body pressure acquisition module and a control circuit. A piezoelectric ceramic array is arranged on the mattress to serve as the BCG signal acquisition module, a resistance-type pressure belt is arranged on the mattress to serve as the human body pressure acquisition module, and in combination with an intelligent dynamic wave peak tracing algorithm, heart rate data, respiration data, body movement data, on-bed/off-bed condition and abnormal sound data of a user in a sleep process can be extracted, and a sleep condition of the user can be comprehensively analyzed. The contactless vital signs monitoring system operates in an intelligent ultralow power consumption operation control mode, thus energy consumption is greatly reduced.

### TECHNICAL FIELD

The invention relates to the field of medical equipment technologies, and particularly to a contactless vital signs monitoring system.

### BACKGROUND

With the development of social economy and technology and the improvement of people's requirements for the quality of life, simplifying and upgrading equipment has become an inevitable requirement of the development of the times, and nowadays, the widespread application of contactless vital signs monitoring technology is one of important manifestations of its development. Long-term continuous vital signs monitoring can enable a health status of a monitored person to be monitored and mastered in real time, but an existing contact type monitoring system uses a wearable device and thus has poor user experience, which not only interferes with the life of the monitored person, but also limits the accuracy of its monitoring effect resulting from that the interference with the life and action of the monitored person would easily cause the monitored person to be nervous and anxious. Therefore, contactless vital signs monitoring technology emerges as the times require, which enables a monitored person to complete physical examination without interference in daily life, and gives suggestions by collecting and analyzing vital signs data, so that the monitored person can avoid bad living habits and prevent chronic diseases. In addition, contactless vital signs monitoring technology can be applied in various fields of life, such as medical treatment, nursing, chronic disease management and health monitoring.

### SUMMARY

A purpose of the invention is to provide a contactless vital signs monitoring system.

Specifically, a contactless vital signs monitoring system according to an embodiment of the invention is suitable for installation on a mattress and includes a contactless vital signs signal acquisition and processing system. The contactless vital signs signal acquisition and processing system includes a ballistocardiography (BCG) signal acquisition module, a human body pressure acquisition module, and a control circuit.

Specifically, the BCG signal acquisition module includes a piezoelectric ceramic array, the piezoelectric ceramic array include several piezoelectric ceramic queues arranged along a lengthwise direction of the mattress, and each of the piezoelectric ceramic queues includes several piezoelectric ceramics sequentially arranged along a widthwise direction of the mattress. The human body pressure acquisition module is disposed on a side of the BCG signal acquisition module facing away from a head of the mattress and configured (i.e., structured and arranged) to detect a pressure applied thereon.

The control circuit is configured to extract heart rate data, respiration data, and abnormal sound signals of a user during rest based on a BCG signal output by the BCG signal acquisition module. A process of the extract is that: first, performing digital filtering on the BCG signal to extract BCG signals with frequencies in ranges of 0.08˜0.5 Hz, 0.66˜3.3 Hz, and 20˜20000 Hz, comparing the BCG signal with the frequency in the range of 0.66˜3.3 Hz with a wave peak regularity of a preset normal electrocardiogram (ECG) signal to extract some features matching a ECG signal model as the heart rate data, comparing the BCG signal with the frequency in the range of 0.08˜0.5 Hz with a wave peak regularity of a preset normal respiration signal to extract some features matching a respiration signal model as the respiration data, and extracting an ambient noise level and the abnormal sound signals including a snoring sound signal, a cough sound signal and a cry for help sound signal from the BCG signal with the frequency in the range of 20˜20000 Hz.

The control circuit is further configured to determine whether the user is on the mattress according to a pressure signal detected by the human body pressure acquisition module, specifically including to: determine the user is on the mattress when the pressure signal is greater than or equal to a preset value, and determine the user is off the mattress when the pressure signal is smaller than the preset value.

The contactless vital signs monitoring system is configured to run at an intelligent ultralow power consumption operation control mode, which includes the control circuit controlling components of the contactless vital signs monitoring system except the human body pressure acquisition module to sleep or shut down when it is determined that the user is off the mattress, and controlling all components of the contactless vital signs monitoring system to normally operate when it is determined the user is on the mattress.

In some embodiments of the invention, the piezoelectric ceramic queues of the piezoelectric ceramic array are two piezoelectric ceramic queues, a distance from a central position of a first one of the two piezoelectric ceramic queues to an end of the head of the mattress is h₁=20 centimeters (cm), and a distance between central positions of the two piezoelectric ceramic queues is h₂=40 cm.

In some embodiments of the invention, the human body pressure acquisition module is embedded in the mattress and includes a resistance-type pressure belt arranged along the widthwise direction of the mattress.

In some embodiments of the invention, the contactless vital signs signal acquisition and processing system further includes a signal filtering amplification module configured to perform filtering and amplification on signals output by the BCG signal acquisition module and the human body pressure acquisition module and transmit to the signals after the filtering and amplification to the control circuit, and the signal filtering amplification module includes a 4T power frequency notch filtering amplification circuit.

In some embodiments of the invention, the control circuit is configured to: according to a characteristic that peaks of the BCG signal are generated by a human body turning over on the mattress, extract signals with the peaks in the BCG signal as a body movement signal of the human body.

In some embodiments of the invention, the snoring sound signal is configured to determine whether the user has an apnea or not, and the ambient noise level is configured to evaluate comfort of a sleeping environment for the user.

In some embodiments of the invention, the control circuit is configured to communicate with a cloud service end through a signal transmitting module of the contactless vital signs signal acquisition and processing system, and the cloud service end is configured to communicate with a monitoring computer and a mobile terminal.

In some embodiments of the invention, the contactless vital signs signal acquisition and processing system further includes a temperature and humidity acquisition module, and the temperature humidity acquisition module includes an integrated digital sensing circuit with a product model of SHT20 and is configured to detect temperature and humidity values of the user in a sleep process.

In some embodiments of the invention, the contactless vital signs monitoring system further includes an environment-friendly green power supply system. The environment-friendly green power supply system is configured to convert energy in an environment of the user into electric energy, and includes: an indoor light energy collection module, an indoor electromagnetic wave collection module, a human body pressure electric energy collection module, a power management circuit, and an electricity storage circuit. The indoor light energy collection module includes a high-efficiency polycrystalline solar cell array and is installed on a head of a bed to collect indoor natural light and lamplight to generate electric energy. The indoor electromagnetic wave collection module includes a ferrite bar antenna and a 2.4 GHz patch antenna, and is configured to collect ambient electromagnetic wave signals to generate electric energy. The human body pressure electric energy collection module includes a piezoelectric film installed on a top surface of the mattress, and is configured to generate electric energy based on a pressure change of the mattress when a human body turns over. Output interfaces of the indoor light energy collection module, the indoor electromagnetic wave collection module and the human body pressure electric energy collection module are connected to the electricity storage circuit and the contactless vital signs signal acquisition and processing system through the power management circuit.

In some embodiments of the invention, the contactless vital signs monitoring system further includes a night getting-up automatic lightning system. The night getting-up automatic lightning system includes an ambient light detector, a night getting-up identification circuit, a night light gradual brightening and darkening module, and a night light. The night getting-up identification circuit and the ambient light detector are both connected to the night light gradual brightening and darkening module. The night light gradual brightening and darkening module is configured to control the night light to gradually brighten or darken. The night getting-up identification circuit is configured to trigger the night light gradual brightening and darkening module to control the night light to gradually brighten when the pressure signal detected by the human body pressure acquisition module indicates the user is off the mattress and an ambient light intensity detected by the ambient light detector is lower than a preset value, and trigger the night light gradual brightening and darkening module to control the night light to gradually darken when the pressure signal detected by the human body pressure acquisition module indicates the user is on the mattress.

Embodiments of the invention can achieve beneficial effects as follows.

1. In some embodiments of the invention, the piezoelectric ceramic array is arranged on the mattress to serve as the BCG signal acquisition module, the resistance-type pressure belt is arranged on the mattress to serve as the human body pressure acquisition module, and in combination with an intelligent dynamic wave peak tracing algorithm, so that heart rate data, respiration data, body movement data, on-bed/off-bed condition and abnormal sound data of a user in a sleep process can be extracted, and a sleep condition of the user can be comprehensively analyzed.

2. The contactless vital signs monitoring system according to some embodiments of the invention is capable of operating in the intelligent ultralow power consumption operation control mode, and when pressure data measured/detected by the human body pressure acquisition module indicates that a user is off the mattress, the contactless vital signs monitoring system turns off most electric components, so that the energy consumption is greatly reduced.

3. The contactless vital signs monitoring system can realize a continuous and stable operation under a condition of being not connected with an external power supply, through the cooperation of various electric energy acquisition modules.

### BRIEF DESCRIPTION OF DRAWINGS

FIG. 1 illustrates a schematic block diagram of a contactless vital signs monitoring system according to some embodiments of the invention.

FIG. 2 illustrates a schematic view of installation of a ballistocardiography (BCG) signal acquisition module and a human body pressure acquisition module on a mattress according to an embodiment of the invention.

### DETAILED DESCRIPTION OF EMBODIMENTS

Embodiments of the invention will be further described in combination with the accompanying drawings.

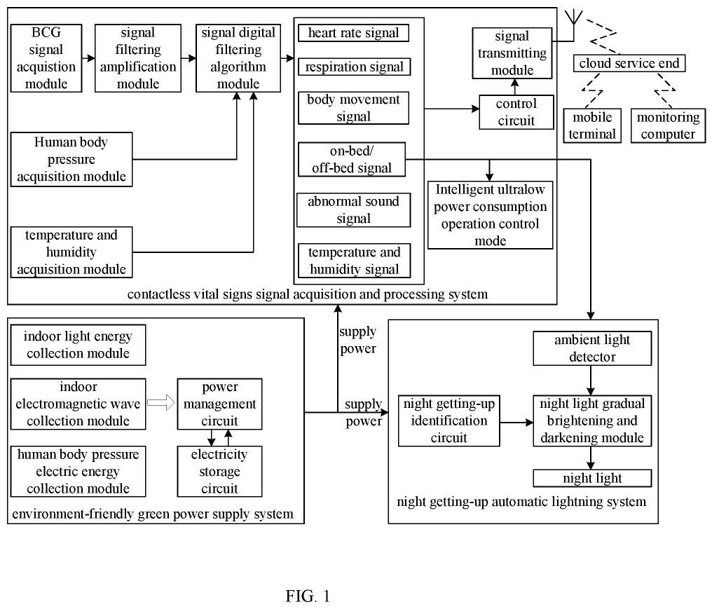

As illustrated in FIG. 1, a contactless vital signs monitoring system is installed on a mattress and includes: a contactless vital signs signal acquisition and processing system, an environment-friendly green power supply system, and a night getting-up automatic lightning system. The contactless vital signs signal acquisition and processing system includes: a BCG signal acquisition module, a human body pressure acquisition module, a signal filtering amplification module, a temperature and humidity acquisition module, a control circuit, and a signal transmitting module (also referred to as signal transmitter).

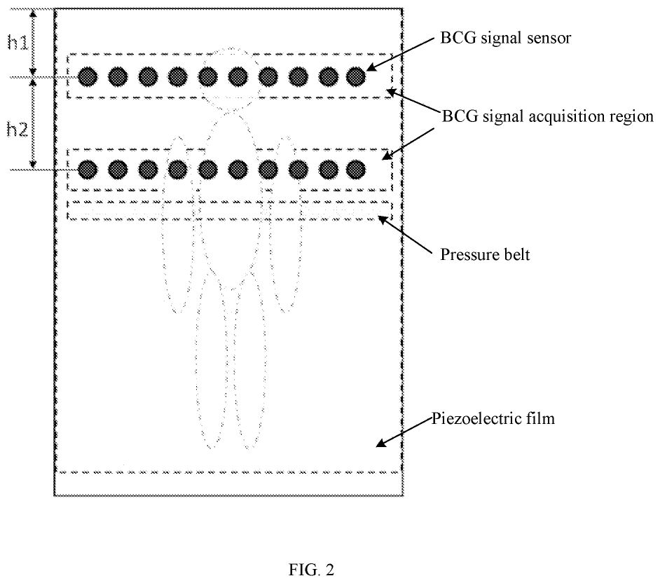

As illustrated in FIG. 1 and FIG. 2, the BCG signal acquisition module includes a piezoelectric ceramic array with high sensitivity. The piezoelectric ceramic array includes several piezoelectric ceramic queues arranged along a lengthwise direction of the mattress. Each piezoelectric ceramic queue includes several piezoelectric ceramics sequentially arranged along a widthwise direction of the mattress. As an illustrated technical solution for further improving the technical effect, the piezoelectric ceramic array is arranged on the mattress with a length of 200 centimeters (cm) and a width of 90 cm, the piezoelectric ceramic array includes two piezoelectric ceramic queues, and a distance from a central position of a first piezoelectric ceramic queue (i.e., the queue near the head of the mattress) to an end of the head of the mattress is h₁=20 cm, a distance between central positions of the two piezoelectric ceramic queues is h₂=40 cm, see FIG. 2 for details. The first piezoelectric ceramic queue is configured (i.e., structured and arranged) to detect a BCG signal a user's head position, and a second piezoelectric ceramic queue (i.e., the other one of the two piezoelectric ceramic queue) is configured to detect a BCG signal at a user's chest position.

The human body pressure acquisition module is disposed on a side of the BCG signal acquisition module facing away from the head of the mattress. The human body pressure acquisition module specifically uses a resistance-type pressure belt. The resistance-type pressure belt is embedded in the mattress and is arranged along the widthwise direction of the mattress. The resistance-type pressure belt can output a certain pressure-induced voltage when being pressed, so as to judge whether a person is on the mattress, see FIG. 2 for details.

The temperature and humidity acquisition module uses an integrated digital sensing circuit e.g., with a product model of SHT20, which can acquire temperature and humidity data of the mattress environment and output them in the form of digital signals. The temperature and humidity signal acquisition module evaluates whether ambient temperature and humidity are in an optimal sleep condition according to appropriate temperature range (preferably, 24° C. to 26° C.) and humidity range (preferably 55% to 65%) of human sleep. The temperature and humidity acquisition module can also output ambient temperature values and perform sleep evaluation in combination with heart rate, breathing, etc.

The signal filtering and amplification module uses 4T (generally an abbreviation of Four T parallel) power frequency notch filtering amplification circuit, which includes an operational amplifier integrated circuit chip and has better clutter elimination effect than a traditional dual-T power frequency notch filter, so that an interference signal in the BCG signal can be well filtered out.

A controller in the control circuit uses an STM32 microcontroller, and the control circuit is configured to: receive BCG signals, a pressure signal, and a temperature and humidity signal which are processed by the signal filtering amplification module; and according to a heart rate signal extraction algorithm, a respiration signal extraction algorithm, a body movement signal extraction algorithm, an on-bed/off-bed signal extraction algorithm, an abnormal sound signal extraction algorithm, and a temperature and humidity signal extraction algorithm, extract heart rate data, respiration data, body movement data, on-bed/off-bed condition, abnormal sound data, and temperature and humidity data of a user during rest as a vital sign signal set.

A specific process of the heart rate signal extraction algorithm is that: first, digital filtering is performed on the acquired BCG signal to extract a BCG signal with a frequency in a range of 0.66 Hz to 3.3 Hz in the acquired BCG signal; and then, an intelligent dynamic wave peak tracing algorithm is used to identify a sinus heart rate signal. The intelligent dynamic wave peak tracing algorithm extracts a wave peak regularity through a normal electrocardiogram (ECG) signal stored in advance to obtain an ECG signal model, then compares the extracted BCG signal with the stored ECG signal model to extract some features of the extracted BCG signal matching the ECG signal model, thereby identifying the heart rate signal, and further can accurately identify BCG signals with different signal amplitudes, thus overcoming defects of a traditional peak extraction algorithm.

A specific process of the respiration signal extraction algorithm is that: first, digital filtering is performed on the acquired BCG signal to extract a BCG signal with a frequency in a range of 0.08 Hz to 0.5 Hz in the acquired BCG signal; and then, the intelligent dynamic wave peak tracing algorithm is used to identify a pulse type respiration signal. The intelligent dynamic wave peak tracing algorithm is to extract a wave peak regularity through a normal respiration signal stored in advance to obtain a respiration signal model, then compares the extracted BCG signal with the stored respiratory signal model to extract some features of the extracted BCG signal matching the respiratory signal model, thereby identifying the respiratory signal, and further can accurately identify BCG signals with different signal amplitudes, thereby overcoming defects of a traditional peak value algorithm.

The body movement signal extraction algorithm specifically is that: based on a characteristic that BCG signal peaks are generated by a human body turning over on the mattress, any signal with the peak is extracted as a body movement signal of the human body.

The on-bed/off-bed signal extraction algorithm specifically is that: according to a characteristic that a human body on the mattress can generate pressure on the human body pressure acquisition module, and based on a pressure received by the human body pressure acquisition module, whether the user is on-bed or off-bed is determined. More specifically, when the human body is on-bed (i.e., generally on the mattress), the pressure belt senses a pressure and generates a pressure value signal (usually the pressure value corresponds to a weight between 10 kg and 30 kg); whereas when the human body is off-bed (i.e., generally off the mattress), the signal of the pressure belt disappears and the pressure value is 0.

A specific process of the abnormal sound signal extraction algorithm is that: first, through a sound frequency range of 20˜20000 Hz set for digital filtering, an ambient noise level and abnormal sound signals including a snoring sound signal, a cough sound signal, and a cry for help sound signal during the user is sleeping are extracted from the acquired BCG signal; the snoring sound signal is used for the system to determine an apnea, so as to be used for an alarm of the apnea and sleep evaluation; the ambient noise level is used to evaluate comfort of a sleeping environment for the user and evaluate impact of background noise on sleep. The other abnormal sound signals including the cough sound signal and the cry for help sound signal are used for alarm and health evaluation.

The control circuit sends out the vital sign signal set in a preset signal encoding format. The signal encoding format includes a data header, a mattress address, heart rate data, respiration data, on-bed/off-bed data, body movement data, abnormal sound data, temperature and humidity data, check bits and a data tail, as shown in TABLE 1.

TABLE 1

| 1 | 2 | 3 | 4 | 5 | 6 | 7 | 8 | 9 | 10 |
|---|---|---|---|---|---|---|---|---|---|
| data | mattress | heart | respiration | on-bed/ | body | abnormal | temperature | check | data |
| header | address | rate | | off-bed | movement | sound | and humidity | bits | tail |
| 8 bits | 8 bits | 8 bits | 8 bits | 4 bits | 4 bits | 16 bits | 16 bits | 8 bits | 8 bits |

In the TABLE 1, the first data are a data starting flag, which is 8-bit data and use a fixed value DD, and the tenth data are a data ending flag, which use a fixed value EE. The rest data are generated based on different mattresses and different signals.

The control circuit communicates with a cloud service end through the signal transmitting module. The signal transmitting module specifically uses five connection modes of WIFI, BLUETOOTH™, 4G, 5G and Ethernet to achieve data transmission between the control circuit and the cloud service end.

The cloud service end uses a cloud server, a vital signs monitoring receiving and management service program is installed in the cloud server, and monitoring data results are stored in the server to provide query and statistical services.

A monitoring computer uses a general desktop computer to access the vital signs monitoring cloud service program by means of a browser, and a mobile terminal accesses the vital signs monitoring cloud service program by means of an APP or a WeChat applet.

The night getting-up automatic lightning system includes an ambient light detector, a night getting-up identification circuit, a night light gradual brightening and darkening module, and a night light. The night getting-up identification circuit determines whether a user gets up or not by monitoring a pressure change of the resistance-type pressure belt. The night light gradual brightening and darkening module includes a time circuit controller, a transition time of the night light gradually brightening is set to be 1 second, and a transition time of the night light gradually darkening is set to be 3-5 seconds, so as to adapt to physiological reaction of the human eyes at night. The night light includes LEDs with low power consumption. When the night getting-up identification circuit detects that the human body leaves the mattress (i.e., off-bed) and the ambient light detector detects that an intensity of ambient light is lower than a preset value, the night light is gradually brightened. When the night getting-up identification circuit detects that the human body is on-bed (i.e., generally on the mattress), the night light is gradually darkened. The night getting-up automatic lightning system can effectively prevent a user from falling down and other hazards caused by getting up at night because they cannot see the environment clearly.

The environment-friendly green power supply system is configured to convert energy in the environment into electric energy and supply power for the contactless vital signs monitoring system. The environment-friendly green power supply system includes an indoor light energy collection module, an indoor electromagnetic wave collection module, a human body pressure electric energy collection module, a power management circuit, and an electricity storage circuit. The indoor light energy collection module uses a high-efficiency polycrystalline solar cell array and is installed on the head of a bed on which the mattress is arranged to collect indoor natural light and lamplight to generate electric energy. The indoor electromagnetic wave collection module includes a ferrite bar antenna and a 2.4 GHz patch antenna, and is configured to collect electromagnetic wave signals generated by a broadcasting station and an indoor WIFI device to generate electric energy. The human body pressure electric energy collection module uses a piezoelectric film installed on a top surface of the mattress, and generates electric energy through a pressure change of the mattress when a human body of the user turns over. The power management circuit exemplarily includes a power management integrated circuit (PMIC), and is connected with the indoor light energy collection module, the indoor electromagnetic wave collection module and the human body pressure electric energy collection module, and performs impedance and power matching and unified allocation management on the electric energy generated by the three collection modules, so as to maximize the efficiency of each collection module. Electric energy output by the power management circuit is supplied to the contactless vital sign monitoring system to work, and redundant electric energy is stored in the electricity storage circuit (exemplarily including lithium battery cells) to supply power at night or when there is no pressure applied by the human body or there is no indoor electromagnetic wave. The environment-friendly green power supply system can effectively overcome problems of electricity safety and fire caused by the use of commercial power, and is green and environment-friendly.

The contactless vital signs monitoring system runs an intelligent ultralow power consumption operation control mode in its working process, and the intelligent ultralow power consumption operation control mode is based on a mattress pressure sensing signal from the resistance-type pressure belt. When no person is detected on the mattress (i.e., the mattress pressure sensing signal is 0), the control circuit controls the rest components in the contactless vital signs monitoring system except the control circuit, the human body pressure acquisition module and the environment-friendly green power supply system to sleep or shut down, so as to reduce the power consumption of the system. When a person is detected on the mattress (i.e., the mattress pressure sensing signal is not 0), the control circuit controls all components in the contactless vital signs monitoring system to work normally. After practical testing, it can be concluded that a person is not on-bed for about ⅔ of the time every day, so that the intelligent ultralow power consumption operation control mode can save ⅔ of power consumption of the contactless vital signs monitoring system, and the intelligent ultralow power consumption operation control mode is unique in contactless vital signs monitoring systems or intelligent mattresses or intelligent monitoring pad products.

What is claimed is:

1. A contactless vital signs monitoring system, being adapted for installation on a mattress and comprising a contactless vital signs signal acquisition and processing system; wherein the contactless vital signs signal acquisition and processing system comprises a ballistocardiography (BCG) signal acquisition module, a human body pressure acquisition module, and a control circuit;
wherein the BCG signal acquisition module comprises a piezoelectric ceramic array, the piezoelectric ceramic array comprises a plurality of piezoelectric ceramic queues arranged along a lengthwise direction of the mattress, each of the plurality of piezoelectric ceramic queues comprises a plurality of piezoelectric ceramics sequentially arranged along a widthwise direction of the mattress, and the human body pressure acquisition module is disposed on a side of the BCG signal acquisition module facing away from a head of the mattress and configured to detect a pressure applied thereon;
wherein the control circuit is configured to extract heart rate data, respiration data, and abnormal sound signals of a user during rest based on a BCG signal output by the BCG signal acquisition module, and a process of the extract is that: first, performing digital filtering on the BCG signal to extract BCG signals with frequencies in ranges of 0.08˜0.5 Hz, 0.66˜3.3 Hz, and 20˜20000 Hz, comparing the BCG signal with the frequency in the range of 0.66˜3.3 Hz with a wave peak regularity of a preset normal electrocardiogram (ECG) signal to extract some features matching a ECG signal model as the heart rate data, comparing the BCG signal with the frequency in the range of 0.08˜0.5 Hz with a wave peak regularity of a preset normal respiration signal to extract some features matching a respiration signal model as the respiration data, and extracting an ambient noise level and the abnormal sound signals including a snoring sound signal, a cough sound signal and a cry for help sound signal from the BCG signal with the frequency in the range of 20˜20000 Hz;
wherein the control circuit is configured to determine whether the user is on the mattress according to a pressure signal detected by the human body pressure acquisition module, specifically including to: determine the user is on the mattress when the pressure signal is greater than or equal to a preset value, and determine the user is off the mattress when the pressure signal is smaller than the preset value;
wherein the contactless vital signs monitoring system is configured to run at an intelligent ultralow power consumption operation control mode, which comprises the control circuit controlling components of the contactless vital signs monitoring system except the human body pressure acquisition module to sleep or shut down when it is determined that the user is off the mattress, and controlling all components of the contactless vital signs monitoring system to normally operate when it is determined the user is on the mattress.

2. The contactless vital signs monitoring system as claimed in claim 1, wherein the plurality of piezoelectric ceramic queues of the piezoelectric ceramic array are two piezoelectric ceramic queues, a distance from a central position of a first one of the two piezoelectric ceramic queues to an end of the head of the mattress is h₁=20 centimeters (cm), and a distance between central positions of the two piezoelectric ceramic queues is h₂=40 cm.

3. The contactless vital signs monitoring system as claimed in claim 1, wherein the human body pressure acquisition module is embedded in the mattress and comprises a resistance-type pressure belt arranged along the widthwise direction of the mattress.

4. The contactless vital signs monitoring system as claimed in claim 1, wherein the contactless vital signs signal acquisition and processing system further comprises a signal filtering amplification module configured to perform filtering and amplification on signals output by the BCG signal acquisition module and the human body pressure acquisition module and transmit to the signals after the filtering and amplification to the control circuit, and the signal filtering amplification module comprises a 4T power frequency notch filtering amplification circuit.

5. The contactless vital signs monitoring system as claimed in claim 1, wherein the control circuit is configured to: according to a characteristic that peaks of the BCG signal are generated by a human body of the user turning over on the mattress, extract signals with the peaks in the BCG signal as a body movement signal of the human body.

6. The contactless vital signs monitoring system as claimed in claim 1, wherein the snoring sound signal is configured to determine whether the user has an apnea or not, and the ambient noise level is configured to evaluate comfort of a sleeping environment for the user.

7. The contactless vital signs monitoring system as claimed in claim 1, wherein the control circuit is configured to communicate with a cloud service end through a signal transmitter of the contactless vital signs signal acquisition and processing system, and the cloud service end is configured to communicate with a monitoring computer and a mobile terminal.

8. The contactless vital signs monitoring system as claimed in claim 1, wherein the contactless vital signs signal acquisition and processing system further comprises a temperature and humidity acquisition module, and the temperature humidity acquisition module comprises an integrated digital sensing circuit with a product model of SHT20 and is configured to detect temperature and humidity values of the user in a sleep process.

9. The contactless vital signs monitoring system as claimed in claim 1, further comprising an environment-friendly green power supply system;
wherein the environment-friendly green power supply system is configured to convert energy in an environment of the user into electric energy, and comprises: an indoor light energy collection module, an indoor electromagnetic wave collection module, a human body pressure electric energy collection module, a power management circuit, and an electricity storage circuit;
wherein the indoor light energy collection module comprises a polycrystalline solar cell array and is installed on a head of a bed to collect indoor natural light and lamplight to generate electric energy;
wherein the indoor electromagnetic wave collection module comprises a ferrite bar antenna and a 2.4 GHz patch antenna, and is configured to collect ambient electromagnetic wave signals to generate electric energy;
wherein the human body pressure electric energy collection module comprises a piezoelectric film installed on a top surface of the mattress, and is configured to generate electric energy based on a pressure change of the mattress when a human body turns over;
wherein output interfaces of the indoor light energy collection module, the indoor electromagnetic wave collection module and the human body pressure electric energy collection module are connected to the electricity storage circuit and the contactless vital signs signal acquisition and processing system through the power management circuit.

10. The contactless vital signs monitoring system as claimed in claim 1, further comprising a night getting-up automatic lightning system;
wherein the night getting-up automatic lightning system comprises an ambient light detector, a night getting-up identification circuit, a night light gradual brightening and darkening module, and a night light;
wherein the night getting-up identification circuit and the ambient light detector are both connected to the night light gradual brightening and darkening module;
wherein the night light gradual brightening and darkening module is configured to control the night light to gradually brighten or darken, and comprises a time circuit controller;
wherein the night getting-up identification circuit is configured to trigger the night light gradual brightening and darkening module to control the night light to gradually brighten when the pressure signal detected by the human body pressure acquisition module indicates the user is off the mattress and an ambient light intensity detected by the ambient light detector is lower than a preset value, and trigger the night light gradual brightening and darkening module to control the night light to gradually darken when the pressure signal detected by the human body pressure acquisition module indicates the user is on the mattress.

11. A contactless vital signs monitoring system, being adapted for installation on a mattress and comprising a contactless vital signs signal acquisition and processing system; wherein the contactless vital signs signal acquisition and processing system comprises a piezoelectric ceramic array for BCG signal acquisition, a pressure belt for human body pressure acquisition, and a control circuit including a microcontroller;
wherein the piezoelectric ceramic array comprises a plurality of piezoelectric ceramic queues arranged along a lengthwise direction of the mattress, each of the plurality of piezoelectric ceramic queues comprises a plurality of piezoelectric ceramics sequentially arranged along a widthwise direction of the mattress, the pressure belt is disposed on a side of the piezoelectric ceramic array facing away from a head of the mattress and configured to detect a pressure applied thereon, and the pressure belt is embedded in the mattress and arranged along the widthwise direction of the mattress;
wherein the control circuit is configured to extract heart rate data, respiration data, and abnormal sound signals of a user during rest based on a BCG signal output by the piezoelectric ceramic array, and a process of the extract is that: first, performing digital filtering on the BCG signal to extract BCG signals with frequencies in ranges of 0.08˜0.5 Hz, 0.66˜3.3 Hz, and 20˜20000 Hz, comparing the BCG signal with the frequency in the range of 0.66˜3.3 Hz with a wave peak regularity of a preset normal electrocardiogram (ECG) signal to extract some features matching a ECG signal model as the heart rate data, comparing the BCG signal with the frequency in the range of 0.08˜0.5 Hz with a wave peak regularity of a preset normal respiration signal to extract some features matching a respiration signal model as the respiration data, and extracting an ambient noise level and the abnormal sound signals including a snoring sound signal, a cough sound signal and a cry for help sound signal from the BCG signal with the frequency in the range of 20˜20000 Hz;
wherein the control circuit is configured to determine whether the user is on the mattress according to a pressure signal detected by the pressure belt, specifically including to: determine the user is on the mattress when the pressure signal is greater than or equal to a preset value, and determine the user is off the mattress when the pressure signal is smaller than the preset value.

12. The contactless vital signs monitoring system as claimed in claim 11, further comprising an environment-friendly green power supply system;
wherein the environment-friendly green power supply system is configured to convert energy in an environment of the user into electric energy, and comprises: an indoor light energy collection module, an indoor electromagnetic wave collection module, a human body pressure electric energy collection module, a power management circuit, and an electricity storage circuit;
wherein the indoor light energy collection module comprises a polycrystalline solar cell array and is installed on a head of a bed on which the mattress is arranged to collect indoor natural light and lamplight to generate electric energy;
wherein the indoor electromagnetic wave collection module comprises a ferrite bar antenna and a 2.4 GHz patch antenna, and is configured to collect ambient electromagnetic wave signals to generate electric energy;
wherein the human body pressure electric energy collection module comprises a piezoelectric film installed on a top surface of the mattress, and is configured to generate electric energy based on a pressure change of the mattress when a human body turns over;
wherein output interfaces of the indoor light energy collection module, the indoor electromagnetic wave collection module and the human body pressure electric energy collection module are connected to the electricity storage circuit and the contactless vital signs signal acquisition and processing system through the power management circuit.

13. The contactless vital signs monitoring system as claimed in claim 12, wherein the plurality of piezoelectric ceramic queues of the piezoelectric ceramic array are two piezoelectric ceramic queues, a distance from a central position of a first one of the two piezoelectric ceramic queues to an end of the head of the mattress is h₁=20 centimeters (cm), and a distance between central positions of the two piezoelectric ceramic queues is h₂=40 cm.
